(19) United States
(12) Patent Application Publication
(10) Pub. No.: US 2021/0269760 A1
(43) Pub. Date: Sep. 2, 2021

(54) All-in-One Distributed and Portable Fermentation Systems with Platform for Holding Same

(71) Applicant: Locus IP Company, LLC, Solon, OH (US)

(72) Inventors: Sean FARMER, Ft. Lauderdale, FL (US); Ken ALIBEK, Solon, OH (US)

(21) Appl. No.: 17/258,292

(22) PCT Filed: Aug. 20, 2019
(86) PCT No.: PCT/US2019/047191

§ 371 Date: Jan. 6, 2021

Related U.S. Application Data
(60) Provisional application No. 62/719,803, filed on Aug. 20, 2018.

(51) Int. Cl. C12M 1/00; C12M 3/00; C12M 1/34; A01N 63/32; C12N 1/14; C12N 1/20; A23K 10/16

(52) U.S. Cl. CPC C12M 23/52; C12M 23/48; C12M 41/12; A23K 10/16; A01N 63/32; C12N 1/14; C12N 1/20; C12M 29/14

(57) ABSTRACT

Systems and methods for producing microbe-based compositions that can be used in the oil and gas industry, environmental cleanup, as well as for other applications are provided. More specifically, a moveable all-in-one system for producing microorganisms and/or metabolites is provided. The system can be delivered to a fermentation site in a ready-to-use state, such that on-site fermentation can be initiated in a short period of time.

### CROSS-REFERENCE TO RELATED APPLICATION

This application claims priority to U.S. Provisional Patent Application No. 62/719,803, filed Aug. 20, 2018, which is incorporated herein by reference in its entirety.

### BACKGROUND OF THE INVENTION

Cultivation of microorganisms such as bacteria, yeast and fungi is important for the production of a wide variety of useful bio-preparations. Microorganisms play crucial roles in, for example, food industries, pharmaceuticals, agriculture, mining, environmental remediation, and waste management.

There exists an enormous potential for the use of microbes in a broad range of industries. An important limiting factor in commercialization of microbe-based products has been the cost per propagule density, where it is particularly expensive and unfeasible to apply microbial products to large scale operations with sufficient inoculum to see the benefits.

Two principle forms of microbe cultivation exist: submerged cultivation and surface cultivation. Bacteria, yeasts and fungi can all be grown using either the surface or submerged cultivation methods. Both cultivation methods require a nutrient medium for the growth of the microorganisms. The nutrient medium, which can either be in a liquid or a solid form, typically includes a carbon source, a nitrogen source, salts and appropriate additional nutrients and microelements. The pH and oxygen levels are maintained at values suitable for a given microorganism.

Microbes have the potential to play highly beneficial roles in, for example, the oil and agriculture industries, if only they could be made more readily available and, preferably, in a more active form.

Oil and natural gas are obtained by drilling into the earth's surface using what is generically referred to as a drilling rig. A well or borehole begins by drilling a large diameter hole (e.g., 24-36 inches in diameter) into the ground using a drill bit.

After the well is drilled, a production liner (or casing) is generally set and the well is then perforated (e.g., explosives are used to puncture the production liner at specific points in the oil bearing formation). Oil then begins to flow out of the well, either under the natural pressure of the formation or by using pressure that is induced via mechanical equipment, water flooding, or other means. As the crude oil flows through the well, substances in the crude oil often collect on the surfaces of the production liners, causing reduction in flow, and sometimes even stopping production all together.

A variety of different chemicals and equipment are utilized to prevent and remediate this issue, but there is a need for improved products and methods. In particular, there is a need for products and methods that are more environmentally friendly, less toxic, and have improved effectiveness.

Similarly to the oil and gas industry, the agriculture industry has relied heavily on the use of synthetic chemicals and chemical fertilizers to boost yields and protect crops against pathogens, pests, and disease; however, when overused or improperly applied, these substances can be air and water pollutants through runoff, leaching and evaporation. Even when properly used, the over-dependence and long-term use of certain chemical fertilizers and pesticides alters soil ecosystems, reduces stress tolerance, increases pest resistance, and impedes plant and animal growth and vitality.

Mounting regulatory mandates governing the availability and use of chemicals, and consumer demands for residue free, sustainably-grown food produced with minimal harm to the environment, are impacting the industry and causing an evolution of thought regarding how to address the myriad of challenges. The demand for safer pesticides and alternate pest control strategies is increasing. While wholesale elimination of chemicals is not feasible at this time, farmers are increasingly embracing the use of biological measures as viable components of Integrated Nutrient Management and Integrated Pest Management programs.

For example, in recent years, biological control of nematodes has caught great interest. This method utilizes biological agents as pesticides, such as live microbes, bio-products derived from these microbes, and combinations thereof. These biological pesticides have important advantages over other conventional pesticides. For example, they are less harmful compared to the conventional chemical pesticides. They are more efficient and specific. They often biodegrade quickly, leading to less environmental pollution.

The use of biopesticides and other biological agents has been greatly limited by difficulties in production, transportation, administration, pricing and efficacy. For example, many microbes are difficult to grow and subsequently deploy to agricultural and forestry production systems in sufficient quantities to be useful. This problem is exacerbated by losses in viability and/or activity due to processing, formulating, storage, and stabilizing prior to distribution. Furthermore, once applied, biological products may not thrive for any number of reasons including, for example, insufficient initial cell densities, the inability to compete effectively with the existing microflora at a particular location, and being introduced to soil and/or other environmental conditions in which the microbe cannot flourish or even survive.

Microbe-based compositions could help resolve some of the aforementioned issues faced by the agriculture industry, the oil and gas industry, as well as many others. Thus, there is a need for more efficient cultivation methods for mass production of microorganisms and microbial metabolites.

### BRIEF SUMMARY OF THE INVENTION

The present invention provides materials, methods and systems for producing microbe-based compositions that can be used in the oil and gas industry, agriculture, health care and environmental cleanup, as well as for a variety of other applications. Specifically, the subject invention provides materials, methods and systems for efficient cultivation of microorganisms and production of microbial growth by-products.

Embodiments of the present invention provide novel systems and methods for producing microorganisms and/or metabolites. An “all-in-one” distributed and moveable system for producing microorganisms and/or metabolites can use submerged fermentation, solid-state fermentation, or both. The system can include a floor portion or platform with shelves or other storage elements disposed thereon. The floor portion can be a moveable platform. The floor portion and storage elements can be enclosed, similar to a train car or a construction trailer, though embodiments are not limited thereto; in certain embodiments, the system can be open-air such that the floor portion is a moveable platform having other elements disposed thereon and not enclosed (either not fully enclosed or not even partially enclosed).

When the system is (at least partially) enclosed, the enclosure can include at least one door or other type of access element. At least one window may also be present, though embodiments are not limited thereto.

The storage elements can have disposed thereon or therein inoculum, nutrient medium, culture cells, and/or other elements used in fermentation to produce microorganisms and/or metabolites.

The system can also include at least one of: a fermentation reactor vessel; a separate vessel for housing nutrient medium; a water tank; a temperature control system; an air compressor; and a mixing system. In some embodiments, the system can include equipment for processing the products of microbial fermentation, for example, a centrifuge, a filtration system, a drying apparatus and/or a blender.

These elements can be situated on the shelves or on the platform, or both. Furthermore, these elements can be connected to each other as appropriate (e.g., via tubing and/or piping). The system can include a sewer connection configured to connect to a sewer system and a water source connection configured to connect to a water source.

Embodiments of the present invention are concerned with “all-in-one” systems that can be delivered to a location and used for producing microorganisms and/or metabolites through fermentation. The system can be ready to use upon delivery, meaning it can be pre-assembled with all elements already in a ready-to-function state. The elements may need to be connected to each other as necessary to produce microorganisms and/or metabolites through fermentation, or they may already be connected to each other as necessary when the system arrives. If a sewer connection and/or water source connection are present, these can be connected to the sewer system or water source as appropriate once the system is delivered to the fermentation location.

In certain embodiments, it may be necessary to provide inoculum, nutrient medium, culture cells, and/or other elements used in fermentation to produce microorganisms and/or metabolites, but once provided these elements can be used as required and/or stored on or in storage elements of the system. In alternative embodiments, inoculum, nutrient medium, culture cells, and/or other elements used in fermentation to produce microorganisms and/or metabolites are already present when the system is delivered, or some elements may be present already and some may be provided once the system is delivered.

Ready-to-use systems of the present invention can advantageously be installed and running (i.e., able to produce microorganisms and/or metabolites (e.g., through fermentation)) on-site in a short period of time (e.g., less than 1 day, less than 12 hours, less than 6 hours, less than 4 hours, less than 2 hours, less than 1 hour, less than 30 minutes, or even less than 15 minutes) after delivery of the system to the location.

In certain embodiments, systems and methods can produce microorganisms and/or metabolites using submerged fermentation. In other embodiments, systems and methods can produce microorganisms and/or metabolites using solid-state fermentation. In some embodiments, a combination of submerged fermentation and solid-state fermentation may be used, though this may result in a larger footprint for the system.

In one embodiment, the subject invention provides methods of cultivating microorganisms. In some embodiments, cultivation occurs without contamination. In certain embodiments, the methods of cultivation comprise adding a culture medium comprising water and nutrient components to the subject system using, for example, a peristaltic pump; inoculating the system with a viable microorganism; and optionally, adding an antimicrobial agent to the culture medium. The antimicrobial agent can be, for example, an antibiotic or a sophorolipid.

In one embodiment, the subject invention further provides a composition comprising at least one type of microorganism and/or at least one microbial metabolite produced by the microorganism that has been grown using the subject fermentation system. The microorganisms in the composition may be in an active or inactive form. The composition may also be in a dried form or a liquid form.

Portability can advantageously result in significant cost savings as microbe-based compositions can be produced at, or near, the site of intended use. This means that the final composition can be manufactured on-site using locally-sourced materials if desired, thereby reducing shipping costs. Furthermore, the compositions can include viable microbes at the time of application, which can increase product effectiveness.

Thus, in certain embodiments, the systems of the subject invention harness the power of naturally-occurring local microorganisms and their metabolic by-products. Use of local microbial populations can be advantageous in settings including, but not limited to, agriculture, environmental remediation (such as in the case of an oil spill), animal husbandry, aquaculture, forestry, pasture management, turf management, horticultural ornamental production, waste disposal and treatment, mining, oil and gas recovery, and human health, including in remote locations.

### BRIEF DESCRIPTION OF THE DRAWINGS

FIG. 1 shows an image of an enclosed system according to an embodiment of the present invention.

FIG. 2 shows two side-view images, with example dimensions, for an enclosed system according to an embodiment of the present invention.

FIG. 3 shows an image of an inside of an enclosed system according to an embodiment of the present invention.

FIG. 4 shows a perspective-view image of an inside of an enclosed system according to an embodiment of the present invention.

### DETAILED DESCRIPTION OF THE INVENTION

The present invention provides materials, methods and systems for producing microbe-based compositions that can be used in the oil and gas industry, agriculture, health care and environmental cleanup, as well as for a variety of other applications. Specifically, the subject invention provides materials, methods and systems for efficient cultivation of microorganisms and production of microbial growth by-products.

Embodiments of the present invention provide novel, low-cost fermentation methods and systems. More specifically, the present invention provides biological reactors (also referred to herein as “systems,” “fermentation systems,” “reactor systems,” and/or “units”) for fermenting a wide variety of, for example, bio level 1 microorganisms with very high cell densities. In specific embodiments, the systems are used to grow yeast- and/or other microbe-based compositions. In certain specific embodiments, the systems can be used for the production of Starmerella bombicola yeast compositions.

The systems can be used to grow yeast, fungi and bacteria. In certain embodiments, the systems can be used for the production of fungi-based and/or yeast-based compositions, including compositions comprising, for example, Trichoderma spp., Starmerella bombicola, Wickerhamomyces anomalus, Meyerozyma guilliermondii and/or Pseudozyma aphidis. These composition can have one or more of the following advantageous properties: high concentrations of mannoprotein and beta-glucan as part of the yeasts' cell wall; and the presence of biosurfactants and other microbial metabolites in the culture.

In some embodiments, the systems can be used for the production of bacteria-based compositions, including compositions comprising, for example, Bacillus spp., Pseudomonas spp. and/or myxobacteria. These compositions can also be beneficial due to the presence of biosurfactants and other microbial metabolites in the culture.

### Selected Definitions

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. In preferred embodiments, the microbes are present, with substrate in which they were grown, in the microbe-based composition. The cells may be absent, or present at, for example, a concentration of 1×10⁴, 1×10⁵, 1×10⁶, 1×10⁷, 1×10⁸, 1×10⁹, 1×10¹⁰, or 1×10¹¹ or more CFU/ml.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “harvested” refers to removing some or all of the microbe-based composition from a growth vessel.

As used herein, a “biofilm” is a complex aggregate of microorganisms, such as bacteria, wherein the cells adhere to each other and/or to a surface. The cells in biofilms are physiologically distinct from planktonic cells of the same organism, which are single cells that can float or swim in liquid medium.

As used herein, the term “control” used in reference to the activity produced by the subject microorganisms extends to the act of killing, disabling or immobilizing pests or otherwise rendering the pests substantially incapable of causing harm.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. As used herein, reference to “isolated” in the context of a microbial strain means that the strain is removed from the environment in which it exists in nature. Thus, the isolated strain may exist as, for example, a biologically pure culture, or as spores (or other forms of the strain) in association with a carrier. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism or a substance necessary for taking part in a particular metabolic process. A metabolite can be an organic compound that is a starting material, an intermediate in, or an end product of metabolism. Examples of metabolites include, but are not limited to, enzymes, acids, solvents, alcohols, proteins, vitamins, minerals, microelements, amino acids, and biosurfactants.

As used herein, “surfactant” refers to a compound that lowers the surface tension (or interfacial tension) between a liquid and a gas, between two liquids or between a liquid and a solid. Surfactants act as, e.g., detergents, wetting agents, emulsifiers, foaming agents, and dispersants. A “biosurfactant” is a surfactant produced by a living organism.

As used herein, “intermediate bulk container,” “IBC” or “pallet tank” refers to a reusable industrial container designed for transporting and storing bulk substances, including, e.g., chemicals (including hazardous materials), food ingredients (e.g., syrups, liquids, granulated and powdered ingredients), solvents, detergents, adhesives, water and pharmaceuticals. Typically, IBCs are stackable and mounted on a pallet designed to be moved using a forklift or a pallet jack. Thus, IBCs are designed to enable portability.

### System Design and Operation

Embodiments of the present invention provide novel systems and method for producing microorganisms and/or metabolites. An “all-in-one” distributed and moveable system for producing microorganisms and/or metabolites can use submerged fermentation, solid-state fermentation, or both. The system can include a floor portion with shelves or other storage elements disposed thereon. The floor portion can be a moveable platform. The floor portion and storage elements can be enclosed, similar to a train car or a construction trailer, though embodiments are not limited thereto.

In certain embodiments, the system can be open-air such that the floor portion is a moveable platform having other elements disposed thereon and not enclosed (either not fully enclosed or not even partially enclosed). When the system is (at least partially) enclosed, the enclosure can include at least one door or other type of access element. At least one window may also be present, though embodiments are not limited thereto. One or more lights can also be present inside the enclosure.

The storage elements can have disposed thereon or therein inoculum, nutrient medium, culture cells, and/or other elements used in fermentation to produce microorganisms and/or metabolites.

The system can also include at least one of: a fermentation reactor vessel; a separate vessel for housing nutrient medium; a water tank; a temperature control system; an air compressor; and a mixing system. In some embodiments, the system also comprises equipment for processing the products of microbial fermentation, such as, for example, a centrifuge, a drying apparatus, a filtration system and/or a blender.

These elements can be situated on the shelves, on the platform floor, or both. Furthermore, these elements can be connected to each other as appropriate (e.g., via tubing and/or piping). The system can include a sewer connection configured to connect to a sewer system and a water source connection configured to connect to a water source.

Embodiments of the present invention are concerned with “all-in-one” systems that can be delivered to a location and used for producing microorganisms and/or metabolites through fermentation. The system can be ready to use upon delivery, meaning it can be pre-assembled with all elements already in a ready-to-function state. The elements may need to be connected to each other as necessary to produce microorganisms and/or metabolites through fermentation, or they may already be connected to each other as necessary when the system arrives. If a sewer connection and/or water source connection are present, these can be connected to the sewer system or water source as appropriate once the system is delivered to the fermentation location.

In certain embodiments, it may be necessary to provide inoculum, nutrient medium, culture cells, and/or other elements used in fermentation to produce microorganisms and/or metabolites, but once provided these elements can be used as required and/or stored on or in storage elements of the system. In alternative embodiments, inoculum, nutrient medium, culture cells, and/or other elements used in fermentation to produce microorganisms and/or metabolites are already present when the system is delivered, or some elements may be present already and some may be provided once the system is delivered.

Ready-to-use systems of embodiments of the present invention can advantageously be installed and running (i.e., able to produce microorganisms and/or metabolites (e.g., through fermentation)) on-site in a short period of time (e.g., less than 1 day, less than 12 hours, less than 6 hours, less than 4 hours, less than 2 hours, less than 1 hour, less than 30 minutes, or even less than 15 minutes) after delivery of the system to the location.

In certain embodiments, systems and methods can produce microorganisms and/or metabolites using submerged fermentation. In other embodiments, systems and methods can produce microorganisms and/or metabolites using solid-state fermentation. In some embodiments, a combination of submerged fermentation and solid-state fermentation may be used, though this may result in a larger footprint for the system.

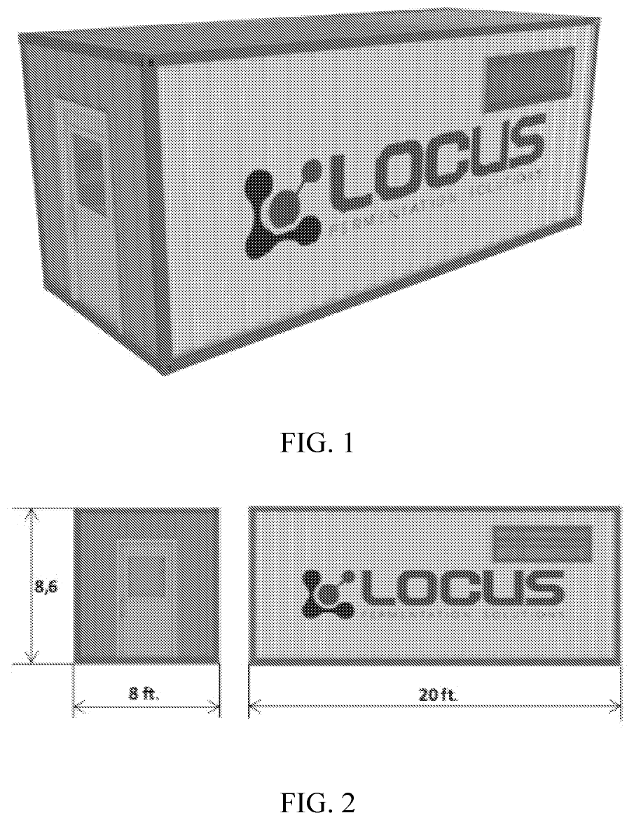

FIG. 1 shows an image of an enclosed system according to an embodiment of the present invention. FIG. 2 shows two images of outer side surfaces of the system of FIG. 1, with example dimensions. Referring to FIGS. 1 and 2, the system can resemble a train car or shipping container and can be completely enclosed. Although FIG. 2 shows the enclosure as 8.5 feet tall, 8 feet wide, and 20 feet long, these dimensions are for exemplary purposes only and should not be construed as limiting.

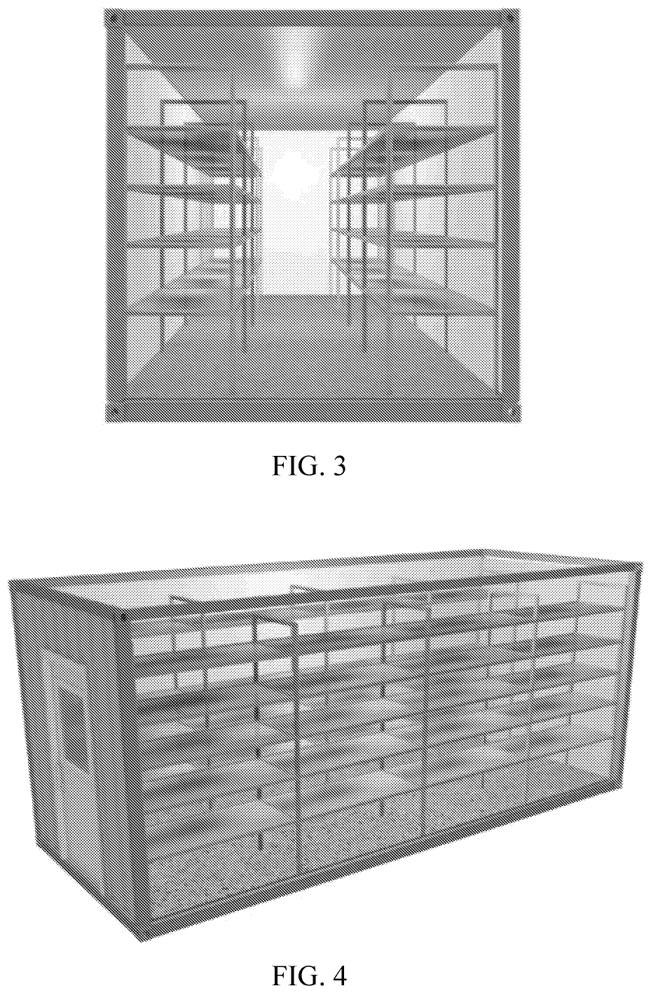

FIG. 3 shows an image of an inside of an enclosed system according to an embodiment of the present invention, and FIG. 4 shows a perspective-view image of the inside of the system shown in FIG. 3. Referring to FIGS. 3 and 4, the system can include a plurality of shelves or other storage elements, which can have reaction elements disposed and/or stored thereon or therein. For example, inoculum, nutrient medium, culture cells, and/or other elements used in fermentation to produce microorganisms and/or metabolites can be stored on or in the storage elements. Furthermore, the fermentation reactor vessel, nutrient medium vessel, water tank and/or other separate elements for carrying out fermentation can be disposed on the shelves, on the platform, or both.

The fermentation vessel(s) used with systems according to the subject invention can be any fermenter or cultivation reactor for industrial use. The vessel can be a tank or another container, such as a bucket, a flask, a tube, a column, or a conical reactor. These vessels may be made of, for example, glass, polymers, metals, metal alloys, and combinations thereof. Preferably, the tank is made of metal, for example, stainless steel.

In a specific embodiment, the system comprises one or more high volume, vertical parallelepiped tanks to serve as the fermentation vessel(s). In one embodiment, the tank is a modified stainless steel intermediate bulk container (“IBC”). Depending upon the oxygen requirements of the fermentation culture, the tank can be formatted as a stirred-tank reactor and/or an unstirred-tank reactor.

Advantageously, the tank or tanks used with the subject systems can be scaled depending on the intended use. For small applications, such as, for example, bioremediation, the reactor tank can be as small as 50 gallons or even smaller. For applications where large volumes of the composition are necessary, such as microbially enhanced oil recovery, the reactor tank can be scaled to produce 20,000 gallons or more of product. In one embodiment, the system can be used as a batch reactor (as opposed to a continuous reactor).

The tank can range in size from a few gallons to tens of thousands of gallons. The tank may be, for example, from 5 liters to 5,000 liters or more. Typically, the tank will be from 10 to 4,000 liters, and preferably from 100 to 2,500 liters.

In one embodiment, the system includes functional controls/sensors configured to measure important factors in the cultivation, such as pH, oxygen, pressure, temperature, agitator shaft power, humidity, viscosity and/or microbial density and/or metabolite concentration.

The system can be equipped with pH stabilization capabilities and temperature controls. The system can also be equipped with an automated system for running a steam sterilization cycle. The system can also be equipped to control dissolved oxygen by cascade to maintain the oxygen levels required for whichever microorganism is being cultivated.

The system can be equipped with an impeller, or mixing motor. In one embodiment, the mixing motor is located at the top of the reactor tank, preferably rotating on a diagonal axis (e.g., an axis at 15 to 60° from vertical).

The reactor tank of the system can comprise an aeration system or an air compressor (e.g., an aeration system or air compressor capable of providing 2 liters of air per liter of culture per minute).

The aeration system can, optionally, have an air filter for preventing contamination of the culture. The aeration system can function to keep the air level over the culture, the DO, and the pressure inside the tank, at desired levels.

In certain embodiments, the reactor tank can be equipped with a sparging system, through which the aeration system supplies air. Preferably, the sparging system comprises sintered stainless steel injectors that produce microbubbles. This allows for proper oxygenation despite the large size of the tank. In an exemplary embodiment, the spargers can comprise sintered stainless steel micro porous (e.g., 2 micron) metal aerators. In one embodiment, the unit requires 1 L/L of inlet air, or approximately 40 CFM of air.

In certain embodiments, the fermentation vessel is a container, such as a tray or pan, that can optionally be sealed with a lid. Solid substrate, such as, for example, corn flour, wheat flour, or other nutrient-rich foodstuffs, can be spread onto the trays, mixed with water, and inoculated with microorganisms as a form of solid state fermentation. A plurality of these tray vessels can be situated on the shelves depicted in FIG. 3.

The system can also be adapted to maintain an appropriate fermentation temperature. For example, the outside of the enclosure and/or the reactor tank can be reflective to avoid raising the system temperature during the day if being operated outdoors. The system can also be insulated (e.g., the reactor tank and/or the enclosure if the system is enclosed can be insulate) so the fermentation process can remain at appropriate temperatures in low temperature environments. Any of the insulating materials known in the art can be applied including fiberglass, silica aerogel, ceramic fiber insulation, etc. The insulation can surround any and/or all of the tubes and/or tanks of the system.

In one embodiment, an external temperature control system is used. In one embodiment, the temperature control system comprises two highly efficient external loops (e.g., with inline 300 to 360K heat exchangers) and circulation pumps (e.g., 1 to 2 hp circulation pumps). The two circulation pumps transport liquid from the bottom of the reactor tank (e.g., at 265 to 270 gallons per minute), through the heat exchangers, and back into the tank at the top of the tank. The heat exchanger can be attached to a chiller, or to an outside water source, whereby the water is pumped (e.g., with a flow rate of about 13 gallons per minute), around the passing culture inside the exchanger, thus increasing or decreasing temperature as desired.

The heat exchanger can utilize an electric heater; however, for larger applications where heat is required, steam or hydrocarbon fuel can be utilized to generate heat. For example, steam input and/or a steam source can be connected to the heat exchanger. The heat exchanger can be a closed system that does not mix water or steam into the reactor.

A thermometer can be included, which can be a manual or automatic thermometer. An automatic thermometer can manage the heat and cooling sources appropriately to control the temperature throughout the fermentation process. The desired temperatures can be programmed on-site or pre-programmed before the system is delivered to the fermentation site. The temperature measurements can then be used to automatically control the temperature control systems that are discussed above.

The pH adjustment can be accomplished by automatic means or it can be performed manually. The automatic pH adjustment can include a pH probe and an electronic device to dispense pH adjustment substances appropriately, depending on the pH measurements. The pH can be set to a specific number by a user or can be pre-programmed to change the pH accordingly throughout the fermentation process. If the pH adjustment is to be performed manually, pH measurement tools known in the art can be included with the system for manual testing.

A computer system for measuring and adjusting of pH and temperature can be used to monitor and control fermentation parameters for the reactor. The computer can be connected to a thermometer and a pH probe, for example. In addition to monitoring and controlling temperature and pH, each vessel may also have the capability for monitoring and controlling, for example, dissolved oxygen, agitation, foaming, purity of microbial cultures, production of desired metabolites and the like. The systems can further be adapted for remote monitoring of these parameters, for example with a tablet, smart phone, or other mobile computing device capable of sending and receiving data wirelessly.

In a further embodiment, the tank or tanks may also be able to monitor the growth of microorganisms inside the reactor tank/vessel (e.g., measurement of cell number and growth phases). Alternatively, a daily sample may be taken from the vessel and subjected to enumeration by techniques known in the art, such as dilution plating technique. Dilution plating is a simple technique used to estimate the number of bacteria in a sample. The technique can also provide an index by which different environments or treatments can be compared.

The reactor tank/vessel can comprise an off-gas system to decrease foam production in the case of submerged fermentation. In some embodiments, the reactor vessel is controlled by a touch screen programmable logic controller (PLC) with a completely automated interface, which can be used to monitor, for example, temperature, DO, and pH throughout fermentation.

In some embodiments, the system further comprises processing equipment, for example, a centrifuge and/or a filtration system for separating components of the products of fermentation; a drying apparatus or an evaporator for lowering and/or removing the moisture content from the products of fermentation; and/or a blender for mixing the fermentation products with additives, such as water or pH adjusters.

Fermentation systems of embodiments of the present invention are mobile and portable and may be provided for on-site production of a microbiological product including a suitable amount of a desired strain of microorganism. Because the microbiological product is generated on-site of the application, without resort to the stabilization, preservation, storage and transportation processes of conventional production, a much higher density of live microorganisms may be generated, thereby requiring a much smaller volume of the microorganism composition for use in the on-site application. This allows for a scaled-down bioreactor (e.g., smaller fermentation tanks, smaller supplies of starter material, nutrients, pH control agents, and de-foaming agent, etc.) that facilitates the mobility and portability of the system.

The system can include a frame for supporting the apparatus components (including the tank(s), flow loops, pumps, etc.). The system can include wheels for moving the apparatus, as well as handles for steering, pushing and pulling when maneuvering the apparatus. The system can include a tow bar or similar attachment for attaching to a truck or other vehicle to be towed to a desired location.

### Microorganisms

The microorganisms grown according to the systems and methods of the subject invention can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a yeast or fungus. Yeast and fungus species suitable for use according to the current invention, include Aspergillus spp, Aureobasidium (e.g., A. pullulans), Blakeslea, Candida (e.g., C. apicola, C. bombicola, C. nodaensis), Cryptococcus, Debaryomyces (e.g., D. hansenii), Entomophthora, Hanseniaspora, (e.g., H. uvarum), Hansenula, Issatchenkia, Kluyveromyces (e.g., K phaffii), Lentinula edodes, Meyerozyma spp. (e.g., M guilliermondii), Mortierella, Mycorrhiza, Phycomyces, Pichia (e.g., P. anomala, P. guilliermondii, P. occidentalis, P. kudriavzevii), Pleurotus spp. (e.g., P. ostreatus), Pseudozyma (e.g., P. aphidis), Saccharomyces (e.g., S. boulardii, S. cerevisiae, S. torula), Starmerella (e.g., S. bombicola), Torulopsis, Trichoderma (e.g., T reesei, T harzianum, T hamatum, T viride), Wickerhamomyces (e.g., W. anomalus), Williopsis (e.g., W. mrakii), Zygosaccharomyces (e.g., Z. bailii), and others.

In certain specific embodiments, the yeast or fungus is, for example, Wickerhamomyces anomalus, Pichia guilliermondii (Meyerozyma guilliermondii), Pichia kudriavzevii (Wickerhamomyces kudriavzevii), Pichia occidentalis, Starmerella bombicola, Pseudozyma aphidis, Lentinula edodes, Pleurotus ostreatus, Trichoderma harzianum, Saccharomyces cerevisiae and/or Saccharomyces boulardii.

The system can also utilize one or more strains of yeast capable of enhancing oil recovery and performing paraffin degradation, e.g., Starmerella (Candida) bombicola, Candida apicola, Candida batistae, Candida floricola, Candida riodocensis, Candida stellate, Candida kuoi, Candida sp. NRRL Y-27208, Rhodotorula bogoriensis sp., Wickerhamiella domericqiae, as well as any other sophorolipid-producing strains of the Starmerella clade. In one embodiment, the microbe is a strain of Starmerella bombicola, for example, American Type Culture Collection (ATCC) accession number 22214. S. bombicola is an effective producer of sophorolipid biosurfactants.

In one embodiment, the microorganisms are bacteria, including gram-positive and gram-negative bacteria. These bacteria may be, but are not limited to, for example, Bacillus (e.g., B. subtilis, B. licheniformis, B. firmus, B. laterosporus, B. megaterium, B. amyloliquefaciens and/or B. coagulans), Clostridium (C. butyricum, C. tyrobutyricum, C. acetobutyricum, and C. beijerinckii), Azotobacter (A. vinelandii, A. chroococcum), Pseudomonas (P. chlororaphis, P. aeruginosa), Azospirillum brasiliensis, Ralslonia eulropha, Rhodospirillum rubrum, Sphingomonas (e.g., S. paucimobilis), Streptomyces (e.g., S. griseochromogenes, S. griseus, S.cacaoi, S. aureus, and S. kasugaenis), Streptoverticillium (e.g., S. rimofaciens), Ralslonia (e.g., R. eulropha), Rhodospirillum (e.g., R. rubrum), Xanthomonas (e.g., X campestris), Erwinia (e.g., E. carotovora), Escherichia coli, Rhizobium (e.g., R. japonicum, Sinorhizobium meliloti, Sinorhizobium fredii, R. leguminosarum biovar trifolii, and R. etli), Bradyrhizobium (e.g., B. japanicum, and B. parasponia), Arthrobacter (e.g., A. radiobacter), Azomonas, Derxia, Beijerinckia, Nocardia, Klebsiella, myxobacteria (e.g.,Myxococcus spp.), Clavibacter (e.g., C. xyli subsp. xyli and C. xyli subsp. cynodontis), Cyanobacteria, Pantoea (e.g., P. agglomerans), and/or Rhodococcus spp. (e.g., R. erythropolis).

In certain specific embodiments, the bacteria is, for example, a Bacillus spp., Pseudomonas spp., Azotobacter spp., Rhodococcus spp., E. coli, and/or a Myxococcus spp.

In one embodiment, the bacteria is a strain of B. subtilis, such as, for example, B. subtilis var. lotuses B1 or B2, which are effective producers of, for example, surfactin and other biosurfactants, as well as biopolymers. This specification incorporates by reference International Publication No. WO 2017/044953 A1 to the extent it is consistent with the teachings disclosed herein.

Other microbial strains including, for example, strains capable of accumulating significant amounts of, for example, biosurfactants, can be used in accordance with the subject invention. Other microbial by-products useful according to the present invention include mannoprotein, beta-glucan, enzymes, solvents, antibiotics, and other metabolites.

In one embodiment, a single type of microbe is grown in a vessel. In alternative embodiments, multiple microbes, which can be grown together without deleterious effects on growth or the resulting product, can be grown in a single vessel. There may be, for example, 2 to 3 or more different microbes grown in a single vessel at the same time.

### Methods of Cultivation Using the Subject Fermentation Systems

In one embodiment, the subject invention provides methods of cultivating microorganisms without contamination using the subject system. In certain embodiments, the methods of cultivation utilize submerged fermentation, and comprise adding a culture medium comprising water and nutrient components to the subject systems using, for example, a peristaltic pump; inoculating the system with a viable microorganism; and optionally, adding an antimicrobial agent to the culture medium. The antimicrobial agent can be, for example, an antibiotic or a sophorolipid.

In certain embodiments, the methods of cultivation utilize solid state fermentation, or hybrid forms and/or modifications thereof. For example, one or more containers can be spread with a solid or semi-solid substrate, such as corn, wheat, soybeans, beans, oats, pasta, and/or flours thereof, mixed with water, and optionally sterilized. This substrate can be inoculated with a sterile liquid nutrient medium pre-seeded with a microorganism and incubated in the enclosure for a number of days.

In one embodiment, the subject invention further provides a composition comprising at least one type of microorganism and/or at least one microbial metabolite produced by the microorganism that has been grown using the subject system. The microorganisms in the composition may be in an active or inactive form. The composition may also be in a dried form or a liquid form. In one embodiment, the composition comprises the microbial metabolite but not the microorganism, where the microorganism or microorganisms are separated from the metabolite(s) and/or other culture medium components.

Prior to microbe growth, the tank(s)/vessel(s) of the system may be disinfected or sterilized. In one embodiment, fermentation medium, air, and equipment used in the method and cultivation process are sterilized. The cultivation equipment such as the reactor/vessel may be separated from, but connected to, a sterilizing unit, e.g., an autoclave. The cultivation equipment may also have a sterilizing unit that sterilizes in situ before starting the inoculation, e.g., by using a steamer. The air can be sterilized by methods know in the art. For example, the ambient air can pass through at least one filter before being supplemented into the vessel. In other embodiments, the medium may be pasteurized or optionally no heat at all added, where the use of low water activity and low pH may be exploited to control unwanted bacterial growth.

Advantageously, the method and system of the subject invention reduce the capital and labor costs of producing microorganisms and their metabolites on a large scale. Furthermore, the cultivation process of the subject invention reduces or eliminates the need to concentrate organisms after completing cultivation. The subject invention provides a cultivation method that not only substantially increases the yield of microbial products per unit of nutrient medium but simplifies production and facilitates portability.

Portability can advantageously result in significant cost savings as microbe-based compositions can be produced at, or near, the site of intended use. This means that the final composition can be manufactured on-site using locally-sourced materials if desired, thereby reducing shipping costs. Furthermore, the compositions can include viable microbes at the time of application, which can increase product effectiveness.

Thus, in certain embodiments, the systems of the subject invention harness the power of naturally-occurring local microorganisms and their metabolic by-products. Use of local microbial populations can be advantageous in settings including, but not limited to, environmental remediation (such as in the case of an oil spill), animal husbandry, aquaculture, forestry, pasture management, turf management, horticultural ornamental production, waste disposal and treatment, mining, oil recovery, and human health, including in remote locations.

The subject invention provides methods and systems for the efficient production of microbes using novel biological reactors. The system can include all of the materials necessary for the fermentation (or cultivation) process, including, for example, equipment, sterilization supplies, and culture medium components, although it is expected that freshwater could be supplied from a local source and sterilized according to the subject methods.

In one embodiment, the system is provided with an inoculum of viable microbes. Preferably, the microbes are biochemical-producing microbes, capable of accumulating, for example, biosurfactants, enzymes, solvents, biopolymers, acids, and/or other useful metabolites. In particularly preferred embodiments, the microorganisms are biochemical-producing yeast (including killer yeasts), fungi, and/or bacteria, including without limitation those listed herein.

In one embodiment, the system is provided with a culture medium. The medium can include nutrient sources, for example, a carbon source, a lipid source, a nitrogen source, and/or a micronutrient source. Each of the carbon source, lipid source, nitrogen source, and/or micronutrient source can be provided in an individual package that can be added to the reactor at appropriate times during the fermentation process. Each of the packages can include several sub-packages that can be added at specific points (e.g., when yeast, pH, and/or nutrient levels go above or below a specific concentration) or times (e.g., after 10 hours, 20 hours, 30 hours, 40 hours, etc.) during the fermentation process.

Before fermentation the reactor vessel can be washed with a hydrogen peroxide solution (e.g., from 2.0% to 4.0% hydrogen peroxide; this can be done before or after a hot water rinse at, e.g., 80-90° C.) to prevent contamination. In addition, or in the alternative, the reactor vessel can be washed with a commercial disinfectant, a bleach solution and/or a hot water or steam rinse. The system can come with concentrated forms of the bleach and hydrogen peroxide, which can later be diluted at the fermentation site before use. For example, the hydrogen peroxide can be provided in concentrated form and be diluted to formulate 2.0% to 4.0% hydrogen peroxide (by weight or volume) for pre-rinse decontamination.

In a specific embodiment, the method of cultivation comprises sterilizing the reactor vessel(s) prior to fermentation. The internal surfaces of the reactor (including, e.g., tanks, ports, spargers and mixing systems) can first be washed with a commercial disinfectant; then fogged (or sprayed with a highly dispersed spray system) with 2% to 4% hydrogen peroxide, preferably 3% hydrogen peroxide; and finally steamed with a portable steamer at a temperature of about 105° C. to about 110° C., or greater.

The culture medium components (e.g., the carbon source, water, lipid source, micronutrients, etc.) can also be sterilized. This can be achieved using temperature decontamination and/or hydrogen peroxide decontamination (potentially followed by neutralizing the hydrogen peroxide using an acid such as HCl, H₂SO₄, etc.).

In a specific embodiment, the water used in the culture medium is UV sterilized using an in-line UV water sterilizer and filtered using, for example, a 0.1-micron water filter. In another embodiment, all nutritional and other medium components can be autoclaved prior to fermentation.

To further prevent contamination, the culture medium of the system may comprise additional acids, antibiotics, and/or antimicrobials, added before, and/or during the cultivation process. The one or more antimicrobial substances can include, e.g., streptomycin, oxytetracycline, sophorolipids, and rhamnolipids.

Inoculation can take place in any and/or all of the reactor tanks (if more than one is present), at which point the inoculum is mixed using through the tubing systems. Total fermentation times can range from 10 to 200 hours, preferably from 20 to 180 hours.

The fermenting temperature utilized in the subject systems and methods can be, for example, from about 25 to 40° C., although the process may operate outside of this range. In one embodiment, the method for cultivation of microorganisms is carried out at about 5° to about 100° C., preferably, 15° to 60° C., more preferably, 25 to 50° C. In a further embodiment, the cultivation may be carried out continuously at a constant temperature. In another embodiment, the cultivation may be subject to changing temperatures.

The pH of the medium should be suitable for the microorganism of interest. Buffering salts, and pH regulators, such as carbonates and phosphates, may be used to stabilize pH near an optimum value. When metal ions are present in high concentrations, use of a chelating agent in the liquid medium may be necessary.

In certain embodiments, the microorganisms can be fermented in a pH range from about 2.0 to about 10.0 and, more specifically, at a p11 range of from about 3.0 to about 7.0 (by manually or automatically adjusting pH using bases, acids, and buffers; e.g., HCl, KOH, NaOH, H₂SO₄, and/or H₃PO₄). The invention can also be practiced outside of this pH range.

The fermentation can start at a first pH (e.g., a pH of 4.0 to 4.5) and later change to a second pH (e.g., a pH of 3.2-3.5) for the remainder of the process to help avoid contamination as well as to produce other desirable results (the first pH can be either higher or lower than the second pH). In some embodiments, pH is adjusted from a first pH to a second pH after a desired accumulation of biomass is achieved, for example, from 0 hours to 200 hours after the start of fermentation, more specifically from 12 to 120 hours after, more specifically from 24 to 72 hours after.

In one embodiment, the moisture level of the culture medium should be suitable for the microorganism of interest. In a further embodiment, the moisture level may range from 20% to 90%, preferably, from 30 to 80%, more preferably, from 40 to 60%.

The cultivation processes of the subject invention can be anaerobic, aerobic, or a combination thereof. Preferably, the process is aerobic, keeping the dissolved oxygen concentration above 10 or 15% of saturation during fermentation, but within 20% in some embodiments, or within 30% in some embodiments.

Advantageously, the system provides easy oxygenation of the growing culture with, for example, slow motion of air to remove low-oxygen containing air and introduction of oxygenated air. The oxygenated air may be ambient air supplemented periodically, such as daily.

Additionally, in the case of submerged fermentation, antifoaming agents can also be added to the system prevent the formation and/or accumulation of foam when gas is produced during cultivation and fermentation.

In one embodiment, the microbe-based composition does not need to be further processed after fermentation (e.g., microbes, metabolites, and remaining nutrients do not need to be separated from the growth by-product of interest, such as biosurfactants). The physical properties of the final product (e.g., viscosity, density, etc.) can also be adjusted using various chemicals and materials that are known in the art.

In one embodiment, the culture medium used in the subject system, may contain supplemental nutrients for the microorganism. Typically, these include carbon sources, proteins, fats, or lipids, nitrogen sources, trace elements, and/or growth factors (e.g., vitamins, pH regulators). It will be apparent to one of skill in the art that nutrient concentration, moisture content, pH, and the like may be modulated to optimize growth for a particular microbe.

The lipid source can include oils or fats of plant or animal origin which contain free fatty acids or their salts or their esters, including triglycerides. Examples of fatty acids include, but are not limited to, free and esterified fatty acids containing from 16 to 18 carbon atoms, hydrophobic carbon sources, palm oil, animal fats, coconut oil, oleic acid, soybean oil, sunflower oil, canola oil, stearic and palmitic acid.

The culture medium of the subject system can further comprise a carbon source. The carbon source is typically a carbohydrate, such as glucose, xylose, sucrose, lactose, fructose, trehalose, galactose, mannose, mannitol, sorbose, ribose, and maltose; organic acids such as acetic acid, fumaric acid, citric acid, propionic acid, malic acid, malonic acid, and pyruvic acid; alcohols such as ethanol, propanol, butanol, pentanol, hexanol, erythritol, isobutanol, xylitol, and glycerol; fats and oils such as canola oil, soybean oil, rice bran oil, olive oil, corn oil, sesame oil, and linseed oil; etc. Other carbon sources can include arbutin, raffinose, gluconate, citrate, molasses, hydrolyzed starch, potato extract, corn syrup, and hydrolyzed cellulosic material. The above carbon sources may be used independently or in a combination of two or more.

In one embodiment, growth factors and trace nutrients for microorganisms are included in the medium of the system. This is particularly preferred when growing microbes that are incapable of producing all of the vitamins they require. Inorganic nutrients, including trace elements such as iron, zinc, potassium, calcium copper, manganese, molybdenum and cobalt; phosphorous, such as from phosphates; and other growth stimulating components can be included in the culture medium of the subject systems. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic or mineral salts may also be included. Inorganic salts can be, for example, potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium carbonate. These inorganic salts may be used independently or in a combination of two or more.

The culture medium of the subject system can further comprise a nitrogen source. The nitrogen source can be, for example, in an inorganic form such as potassium nitrate, ammonium nitrate, ammonium sulfate, ammonium phosphate, ammonia, urea, and ammonium chloride, or an organic form such as proteins, amino acids, yeast extracts, yeast autolysates, corn peptone, casein hydrolysate, and soybean protein. These nitrogen sources may be used independently or in a combination of two or more.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

### Preparation of Microbe-Based Products

The microbe-based products of the subject invention include products comprising the microbes and/or microbial growth by-products and optionally, the growth medium and/or additional ingredients such as, for example, water, carriers, adjuvants, nutrients, viscosity modifiers, and other active agents.

One microbe-based product of the subject invention is simply the fermentation medium containing the microorganism and/or the microbial growth by-products produced by the microorganism and/or any residual nutrients. The product of fermentation may be used directly without extraction or purification. If desired, extraction and purification can be easily achieved using standard extraction methods or techniques known to those skilled in the art.

The microorganisms in the microbe-based products may be in an active or inactive form and/or in the form of vegetative cells, spores, mycelia, conidia and/or any form of microbial propagule. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium resulting from the microbial growth can be removed from the growth vessel and transferred via, for example, piping for immediate use.

In other embodiments, the composition (microbes, medium, or microbes and medium) can be placed in containers of appropriate size, taking into consideration, for example, the intended use, the contemplated method of application, the size of the fermentation tank, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, nutrients for plant growth, tracking agents, pesticides, herbicides, animal feed, food products and other ingredients specific for an intended use.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

The microbe-based products of the subject invention may be, for example, microbial inoculants, biopesticides, nutrient sources, remediation agents, health products, and/or biosurfactants.

In one embodiment, the fermentation products (e.g., microorganisms and/or metabolites) obtained after the cultivation process are typically of high commercial value. Those products containing microorganisms have enhanced nutrient content than those products deficient in the microorganisms. The microorganisms may be present in the cultivation system, the cultivation broth and/or cultivation biomass. The cultivation broth and/or biomass may be dried (e.g., spray-dried), to produce the products of interest.

In one embodiment, the cultivation products may be prepared as a spray-dried biomass product. The biomass may be separated by known methods, such as centrifugation, filtration, separation, decanting, a combination of separation and decanting, ultrafiltration or microfiltration. The biomass cultivation products may be further treated to facilitate rumen bypass. The biomass product may be separated from the cultivation medium, spray-dried, and optionally treated to modulate rumen bypass, and added to feed as a nutritional source.

In one embodiment, the cultivation products may be used as an animal feed or as food supplement for humans. The cultivation products may be rich in at least one or more of fats, fatty acids, lipids such as phospholipid, vitamins, essential amino acids, peptides, proteins, carbohydrates, sterols, enzymes, and trace minerals such as, iron, copper, zinc, manganese, cobalt, iodine, selenium, molybdenum, nickel, fluorine, vanadium, tin and silicon. The peptides may contain at least one essential amino acid.

In other embodiments, the essential amino acids are encapsulated inside a subject modified microorganism used in a cultivation reaction. The essential amino acids are contained in heterologous polypeptides expressed by the microorganism. Where desired, the heterologous peptides are expressed and stored in the inclusion bodies in a suitable microorganism (e.g., fungi).

In one embodiment, the cultivation products have a high nutritional content. As a result, a higher percentage of the cultivation products may be used in a complete animal feed. In one embodiment, the feed composition comprises the modified cultivation products ranging from 15% of the feed to 100% of the feed.

The subject invention further provides materials and methods for the production of biomass (e.g., viable cellular material), extracellular metabolites (e.g., both small and large molecules), and/or intracellular components (e.g., enzymes and other proteins). The microbes and microbial growth by-products of the subject invention can also be used for the transformation of a substrate, such as an ore, wherein the transformed substrate is the product.

The subject invention further provides microbe-based products, as well as uses for these products to achieve beneficial results in many settings including, for example, improved bioremediation, mining, and oil and gas production; waste disposal and treatment; enhanced health of livestock and other animals; and enhanced health and productivity of plants by applying one or more of the microbe-based products.

In specific embodiments, the systems of the subject invention provide science-based solutions that improve agricultural productivity by, for example, promoting crop vitality; enhancing crop yields; enhancing plant immune responses; enhancing insect, pest and disease resistance; controlling insects, nematodes, diseases and weeds; improving plant nutrition; improving the nutritional content of agricultural and forestry and pasture soils; and promoting improved and more efficient water use.

In one embodiment, the subject invention provides a method of improving plant health and/or increasing crop yield by applying the composition disclosed herein to soil, seed, or plant parts. In another embodiment, the subject invention provides a method of increasing crop or plant yield comprising multiple applications of the composition described herein.

Advantageously, the method can effectively control nematodes, and the corresponding diseases caused by pests while a yield increase is achieved and side effects and additional costs are avoided.

In another embodiment, the method for producing microbial growth by-products may further comprise steps of concentrating and purifying the by-product of interest.

In one embodiment, the subject invention further provides a composition comprising at least one type of microorganism and/or at least one microbial growth by-product produced by said microorganism. The microorganisms in the composition may be in an active or inactive form and/or in the form of vegetative cells, spores, mycelia, conidia and/or any form of microbial propagule. The composition may or may not comprise the growth matrix in which the microbes were grown. The composition may also be in a dried form or a liquid form.

In one embodiment, the composition is suitable for agriculture. For example, the composition can be used to treat soil, plants, and seeds. The composition may also be used as a pesticide.

In one embodiment, the subject invention further provides customizations to the materials and methods according to the local needs. For example, the method for cultivation of microorganisms may be used to grow those microorganisms located in the local soil or at a specific oil well or site of pollution. In specific embodiments, local soils may be used as the solid substrates in the cultivation method for providing a native growth environment. Advantageously, these microorganisms can be beneficial and more adaptable to local needs.

The cultivation method according to the subject invention not only substantially increases the yield of microbial products per unit of nutrient medium but also improves the simplicity of the production operation. Furthermore, the cultivation process can eliminate or reduce the need to concentrate microorganisms after finalizing fermentation.

Advantageously, the method does not require complicated equipment or high energy consumption, and thus reduces the capital and labor costs of producing microorganisms and their metabolites on a large scale.

### Microbial Growth By-Products

The methods and systems of the subject invention can be used to produce useful microbial growth by-products such as, for example, biosurfactants, enzymes, acids, biopolymers, solvents, and/or other microbial metabolites. In specific embodiments, the growth by-product is a biosurfactant.

Biosurfactants are a structurally diverse group of surface-active substances produced by microorganisms. Biosurfactants are biodegradable and can be produced using selected organisms on renewable substrates. Most biosurfactant-producing organisms produce biosurfactants in response to the presence of a hydrocarbon source (e.g., oils, sugar, glycerol, etc.) in the growing media.

All biosurfactants are amphiphiles. They consist of two parts: a polar (hydrophilic) moiety and non-polar (hydrophobic) group. Due to their amphiphilic structure, biosurfactants increase the surface area of hydrophobic water-insoluble substances, increase the water bioavailability of such substances, and change the properties of bacterial cell surfaces. The common lipophilic moiety of a biosurfactant molecule is the hydrocarbon chain of a fatty acid, whereas the hydrophilic part is formed by ester or alcohol groups of neutral lipids, by the carboxylate group of fatty acids or amino acids (or peptides), by an organic acid in the case of flavolipids, or, in the case of glycolipids, by a carbohydrate.

Biosurfactants include low molecular weight glycolipids (e.g., rhamnolipids, sophorolipids, trehalose lipids, cellobiose lipids and mannosylerythritol lipids), lipopeptides (e.g., surfactin, lichenysin, fengycin, arthrofactin, viscosin, and iturin), flavolipids, fatty acid esters, phospholipids (e.g., cardiolipin), and high molecular weight polymers such as lipoproteins, lipopolysaccharide-protein complexes, and polysaccharide-protein-fatty acid complexes.

Microbial biosurfactants are produced by a variety of microorganisms such as bacteria, fungi, and yeasts. Exemplary biosurfactant-producing microorganisms include Pseudomonas species (P. aeruginosa, P. putida, P. florescens, P. fragi, P. syringae); Flavobacterium spp.; Bacillus spp. (B. subtilis, B. pumillus, B. cereus, B. licheniformis); Wickerhamomyces spp., Candida spp. (C. albicans, C. rugosa, C. tropicalis, C. lipolytica, C. torulopsis); Rhodococcus spp.; Arthrobacter spp.; Campylobacter spp.; Cornybacterium spp.; Pichia spp.; Starmerella spp.; and so on.

In one embodiment of the subject invention, the biosurfactants produced by the subject systems include surfactin and glycolipids such as rhamnolipids (RLP), sophorolipids (SLP), trehalose lipids or mannosylerythritol lipids (MEL). In particular embodiments, the subject system is used to produce SLP and/or MEL on a large scale.

Sophorolipids are glycolipid biosurfactants produced by, for example, various yeasts of the Starmerella clade. Among yeasts of the Starmerella clade that have been examined, the greatest yield of sophorolipids has been reported from Candida apicola and Starmerella bombicola. SLPs consist of a disaccharide sophorose linked to long chain hydroxy fatty acids. These SLPs are a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid is generally 16 or 18 carbon atoms, and may contain one or more unsaturated bonds. The fatty acid carboxyl group can be free (acidic or open form) or internally esterified at the 4″-position (lactone form).

Mannosylerythritol lipids are a glycolipid class of biosurfactants produced by a variety of yeast and fungal strains. Effective MEL production is limited primarily to the genus Pseudozyma, with significant variability among the MEL structures produced by each species. MELs contain 4-C-b-D-mannopyranosyl-erythritol as their sugar moiety or a hydrophilic unit. According to the degree of acetylation at C-4′ and C-6′positions in mannopyranosyl, MELs are classified as MEL-A, MEL-B, MEL-C and MEL-D. MEL-A represents the diacetylated compound whereas MEL-B and MEL-C are monoacetylated at C-6′and C-4′, respectively. The completely deacetylated structure is attributed to MEL-D. Outside of Pseudozyrna, a recently isolated strain, Ustilago scitaminea, has been shown to exhibit abundant MEL-B production from sugarcane juice. MELs act as effective topical moisturizers and can repair damaged hair. Furthermore, these compounds have been shown to exhibit both protective and healing activities, to activate fibroblasts and papilla cells, and to act as natural antioxidants.

Due to the structure and composition of SLPs and MELs, these biosurfactants have excellent surface and interfacial tension reduction properties, as well as other beneficial biochemical properties, which can be useful in applications such as large scale industrial and agriculture uses, and in other fields, including but not limited to cosmetics, household products, and health, medical and pharmaceutical fields.

Biosurfactants accumulate at interfaces, thus reducing interfacial tension and leading to the formation of aggregated micellar structures in solution. Safe, effective microbial biosurfactants reduce the surface and interfacial tensions between the molecules of liquids, solids, and gases. The ability of biosurfactants to form pores and destabilize biological membranes permits their use as antibacterial, antifungal, and hemolytic agents. Combined with the characteristics of low toxicity and biodegradability, biosurfactants are advantageous for use in the oil and gas industry for a wide variety of petroleum industry applications, such as microbially enhanced oil recovery. These applications include, but are not limited to, enhancement of crude oil recovery from an oil-containing formation; stimulation of oil and gas wells (to improve the flow of oil into the well bore); removal of contaminants and/or obstructions such as paraffins, asphaltenes and scale from equipment such as rods, tubing, liners, tanks and pumps; prevention of the corrosion of oil and gas production and transportation equipment; reduction of H₂S concentration in crude oil and natural gas; reduction in viscosity of crude oil; upgradation of heavy crude oils and asphaltenes into lighter hydrocarbon fractions; cleaning of tanks, flowlines and pipelines; enhancing the mobility of oil during water flooding though selective and non-selective plugging; and fracturing fluids.

When used in oil and gas applications, the systems of the present invention can be used to lower the cost of microbial-based oilfield compositions and can be used in combination with other chemical enhancers, such as polymers, solvents, fracking sand and beads, emulsifiers, surfactants, and other materials known in the art.

Biosurfactants produced according to the subject invention can be used for other, non-oil recovery purposes including, for example, cleaning pipes, reactors, and other machinery or surfaces, as well as pest control, for example, when applied to plants and/or their surrounding environment. Some biosurfactants produced according to the subject invention can be used to control pests because they are able to penetrate through pests' tissues and are effective in low amounts without the use of adjuvants. It has been found that at concentrations above the critical micelle concentration, the biosurfactants are able to penetrate more effectively into treated objects.

Pests can be controlled using either the biosurfactant-producing organisms as a biocontrol agent or by the biosurfactants themselves. In addition, pest control can be achieved by the use of specific substrates to support the growth of biosurfactant-producing organisms as well as to produce biosurfactant pesticidal agents. Advantageously, natural biosurfactants are able to inhibit the growth of competing organisms and enhance the growth of the specific biosurfactant-producing organisms.

In addition, these biosurfactants can play important roles in treating animal and human diseases. Animals can be treated by, for example, by dipping or bathing in a biosurfactant solution alone, with or without microbe cell mass, and/or in the presence of other compounds such as copper or zinc.

The compositions produced according to the present invention have advantages over biosurfactants alone due to the use of entire cell culture, including: high concentrations of mannoprotein as a part of yeast cell wall's outer surface (mannoprotein is a highly effective bioemulsifier capable of reaching up to an 80% emulsification index); the presence of the biopolymer beta-glucan (an emulsifier) in yeast cell walls; the presence of sophorolipids in the culture, which is a powerful biosurfactant capable of reducing both surface and interfacial tension; and the presence of metabolites (e.g., lactic acid, ethanol, etc.) in the culture. These compositions can, among many other uses, act as biosurfactants and can have surface/interfacial tension-reducing properties.

Cultivation of microbial biosurfactants according to the prior art is a complex, time and resource consuming, process that requires multiple stages. The subject invention provides equipment, apparatuses, methods and systems that simplify and reduce the cost of this process. The subject invention also provides novel compositions and uses of these compositions.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1

### Enclosed System for Producing Microorganisms and/or Metabolites

FIGS. 1-4 show images of an enclosed system for producing microorganisms and/or metabolites. The system includes a floor portion with shelves disposed thereon. The floor portion and shelves are enclosed, similar to a train car or a shipping container. A door and window are included on the enclosure, and a light is present inside the enclosure.

Though not shown, the system can include inoculum, nutrient medium, culture cells, and/or other elements used in fermentation to produce microorganisms and/or metabolites. The system can also include (not shown) at least one of: a fermentation reactor vessel; a separate vessel for housing nutrient medium; a water tank; a temperature control system; an air compressor; and a mixing system. These elements can be connected to each other as appropriate (e.g., via tubing and/or piping).

The system can include a sewer connection configured to connect to a sewer system and a water source connection configured to connect to a water source.

The enclosure can also include wheels (not shown) and a tow bar or other attachment for attaching to a vehicle to be transported.

### Example 2

### Solid State Fermentation of Fungal and Bacterial Spores

For growing Trichoderma spp., 250 g of nixtamilized corn flour are mixed with deionized water and sterilized in a stainless steel steam pan, then sealed with a lid and pan bands. These pan reactor vessels with corn flour media are aseptically inoculated with Trichoderma seed culture. The pans are then placed on the shelves depicted in FIG. 3. If desired, all of the shelves can be filled with these pans to enable increased production capabilities.

The pans are then incubated in the enclosed system at 30° C. for 10 days. After 10 days, the flour substrate, Trichoderma spores and any growth by-products thereof can be blended, milled and/or micronized, and optionally dried. The final dry product can contain, for example, 1×10⁹ spores/g or more of Trichoderma propagules.

For Bacillus spp. spore production, a wheat bran-based media is used. The media is sterilized in stainless steel steam pans, then sealed with a lid and pan bands. Following sterilization, the pans are inoculated with seed culture and incubated in the enclosure of the subject system for 48-72 hours. At the end of fermentation, the flour substrate, Bacillus spores and any growth by-products thereof can be blended, milled and/or micronized, and optionally dried. The final dry product can contain, for example, 1×10¹⁰ spores/g or more of Bacillus.

It should be understood that the examples and embodiments described herein are for illustrative purposes only and that various modifications or changes in light thereof will be suggested to persons skilled in the art and are to be included within the spirit and purview of this application. AH patents, patent applications, provisional applications, and publications referred to or cited herein are incorporated by reference in their entirety, including all figures and tables, to the extent they are not inconsistent with the explicit teachings of this specification.

1. A moveable system for producing microorganisms, metabolites, or both, the system comprising:
a platform;
a fermentation reactor vessel disposed on the platform; and
an enclosure surrounding and enclosing the platform and the fermentation reactor vessel,
wherein the system is configured to be transported from a first location to a second location different from the first location.

2. The system of claim 1, further comprising a water source connection configured to be connected to a water source.

3. The system of claim 1, further comprising a sewer connection configured to be connected to a sewer system.

4. The system of claim 1, further comprising a nutrient medium vessel for housing nutrient medium, the nutrient medium vessel being connected to the fermentation reactor vessel via tubing, piping, or both.

5. The system of claim 1, further comprising a temperature control system connected to the fermentation reactor vessel and configured to control a temperature within the fermentation reactor vessel.

6. (canceled)

7. The system of claim 1, further comprising an air compressor connected to the femientation reactor vessel and configured to supply air to the fermentation reactor vessel.

8. The system of claim 1, configured to produce microorganisms, metabolites, or both using submerged fermentation.

9. The system of claim 1, configured to produce microorganisms, metabolites, or both using solid-state fermentation.

10. (canceled)

11. The system of claim 1, wherein the enclosure is at least 6 feet tall, at most 8 feet wide, and at most 20 feet long.

12-13. (canceled)

14. The system of claim 1, wherein the enclosure comprises a door.

15-16. (canceled)

17. The system of claim 1, further comprising a tow attachment configured to attach to a vehicle such that the system can be towed by the vehicle.

18. The system of claim 1, further comprising wheels.

19. A method for producing microorganisms, wherein said method comprises:
adding a culture medium comprising water and nutrient components to the fermentation reactor vessel of the system of claim 1;
inoculating the fermentation reactor vessel with a viable microorganism; and
optionally, adding an antimicrobial agent to the fermentation reactor vessel.

20. The method of claim 19, wherein adding the culture medium comprises adding the culture medium using a peristaltic pump.

21. The method of claim 19, wherein the microorganism is a yeast and/or fungus.

22. The method of claim 19, wherein the microorganism is Starmerella bombicola, Meyerozyma guilliermondii, Pseudozyma aphidis, Wickerhamomyces anomalus, or a Trichoderma spp.

23. The method of claim 19, wherein the microorganism is a Bacillus spp. bacteria selected from B. subtilis, B. licheniformis, and B. amyloliquefaciens.

24-38. (canceled)

39. A method for improving plant growth, yield, and/or health, wherein said method comprises applying to the plant or its environment a composition comprising a yeast produced by the method of claim 19 and/or a biosurfactant produced by the yeast.

40. (canceled)

41. A method for feeding an animal, wherein the method comprises adding the composition comprising a yeast produced by the method of claim 19 and/or a biosurfactant produced by the yeast to the animal's food and/or drinking water source.
